(19) United States
(12) Patent Application Publication
(10) Pub. No.: US 2025/0248910 A1
(43) Pub. Date: Aug. 7, 2025

(54) COSMETIC COMPOSITIONS FOR LIPS COMPRISING COLORANTS

(71) Applicant: AMOREPACIFIC CORPORATION, Seoul (KR)

(72) Inventors: Serim YOON, Yongin-si (KR); Jungsun CHO, Yongin-si (KR); Eunsil HAN, Yongin-si (KR)

(73) Assignee: AMOREPACIFIC CORPORATION, Seoul (KR)

(21) Appl. No.: 19/043,831

(22) Filed: Feb. 3, 2025

(30) Foreign Application Priority Data
Feb. 6, 2024 (KR) 10-2024-0018193

(51) Int. Cl. A61K 8/29; A61Q 1/06

(52) U.S. Cl. CPC A61K 8/29; A61Q 1/06; A61K 2800/43; A61K 2800/592

(57) ABSTRACT

A lip cosmetic composition including a colorant, specifically an aryl-based green colorant; and titanium dioxide coated with a hydrophilic compound; wherein the composition can achieve lip colors of various brightness and saturation, demonstrate excellent staining effects, while minimizing darkening and yoplait phenomena on the lips.

### CROSS REFERENCE TO RELATED APPLICATION

The present application claims priority to Korean Patent Application No. 10-2024-0018193, filed Feb. 6, 2024, the entire contents of which are hereby incorporated by this reference.

### FIELD OF THE INVENTION

The present disclosure relates to a lip cosmetic composition comprising a colorant.

### BACKGROUND

Conventional lip cosmetics have primarily consisted of products with high saturation colors. However, recently, with the increasing interest in various colors, there has been a growing demand for low-saturation, toned-down colors. To satisfy this demand, a blue colorant may be used to tone down the color, or titanium dioxide may be used to impart a whitish opacity.

However, blue colorants can only achieve a cool, blue color and, due to their inherent color hues, may cause darkening, which can result in making the color of the lips appear dull. In addition, titanium dioxide is prone to precipitation and aggregation in the aqueous phase of the formulation, which may lead to poor dispersion and quality issues. Further, when titanium dioxide is used together with a colorant, the colorant and titanium dioxide may not interact properly, leading to a phenomenon where they become a messy tangle on the lips (Yoplait phenomenon, This can also be referred to as the “white ring”), which does not allow for maximum color development and staining effects.

Therefore, it is necessary to develop a lip cosmetic that can implement a various range of colors, maximizes the staining effect when applied to the lips, while minimizing both darkening and the yoplait phenomenon.

### SUMMARY OF THE INVENTION

One aspect of the present disclosure is directed to providing a lip cosmetic composition comprising a colorant.

One aspect of the present disclosure is directed to providing a method for lip makeup.

A lip cosmetic composition according to one aspect of the present disclosure includes an aryl-based green colorant; and titanium dioxide (TiO₂) coated with a hydrophilic compound.

A method for lip makeup according to one aspect of the present disclosure, includes: applying the lip cosmetic composition according to one aspect.

The lip cosmetic composition according to one aspect of the present disclosure can achieve lip colors of various brightness and saturation, demonstrate excellent staining effects, and simultaneously minimize darkening and yoplait phenomena (a phenomenon where the formulation separates and becomes whitish on the lips, which can occur when titanium dioxide is used).

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1 illustrates the dispersibility of a cosmetic composition according to an embodiment.

FIG. 2 illustrates the formulation stability and the occurrence of the yoplait phenomenon on the lips for a cosmetic composition including hydrophilic compound-coated titanium dioxide and a cosmetic composition including silica-complexed titanium dioxide according to an embodiment.

FIG. 3 illustrates the color and stained color of a cosmetic composition including a green colorant and a cosmetic composition including a blue colorant, according to an embodiment.

FIG. 4 is a graph showing the results of brightness measurements using a colorimeter for a cosmetic composition including a green colorant and a cosmetic composition including a blue colorant according to an embodiment.

FIG. 5 illustrates the colors implemented by mixing a green colorant and a blue colorant with a red colorant, respectively, according to an embodiment.

### DETAILED DESCRIPTION

Various embodiments of the present document and the terms used in the embodiments are not intended to limit the technical features disclosed in the present document to the particular embodiments and should be understood as including various alterations, equivalents, or alternatives of the corresponding embodiments.

One aspect of the present disclosure provides a lip cosmetic composition including a green colorant and titanium dioxide (TiO₂) coated with a hydrophilic compound.

“Colorant” may refer to a substance added to food, pharmaceuticals, or cosmetics for the purpose of coloring, and may include, for example, a dye. For example, the colorant may be a natural or synthetic colorant. For example, the colorant may be an organic or inorganic substance.

In one embodiment, the green colorant may be a green dye, but is not limited thereto.

In one embodiment, the green colorant may be at least one selected from the group consisting of green organic dyes and green inorganic dyes, but is not limited thereto.

In one embodiment, the green colorant may be an aryl-based green colorant.

The term “aryl-based” compound may refer to a compound that includes an aryl. The term “aryl” may refer to a fully or partially unsaturated substituted or unsubstituted monocyclic or polycyclic carbon ring. Aryl may include fused aryl. Aryl may be substituted or unsubstituted.

“Fused aryl” may refer to a ring structure consisting of fused multiple aryl rings, and may include, for example, naphthalene, phenanthrene, anthracene, benzopyrene, acenaphthylene, acenaphthene, chrysene, fluoranthene, fluorene, pyrene, and the like.

In one embodiment, the aryl-based green colorant may be at least one selected from the group consisting of anthraquinone-based green pigments, triphenylmethane-based green pigments, and pyrene-based green pigments, but is not limited thereto.

In one embodiment, the aryl-based green colorant may be at least one selected from the group consisting of Green No. 3 (CI number 42053), Green No. 201 (CI number 61570), Green No. 202 (CI number 61565), and Green No. 204 (CI number 59040), but is not limited thereto.

A cosmetic composition according to one aspect of the present disclosure may achieve a coloring effect that is not dull after staining by including a green colorant, and darkening may not occur significantly even after a predetermined period of time following staining.

A cosmetic composition according to one aspect of the present disclosure may achieve colors of various brightness and saturation, which are difficult to implement with a blue colorant, by including a green colorant. According to an embodiment, when a green colorant is used together with additional colorant (e.g., a red colorant), colors of various brightness and saturation may be achieved. Meanwhile, when a blue colorant is used together with the other colorant (e.g., a red colorant), even a small amount of the blue colorant may significantly reduce the brightness, making it relatively difficult to achieve a variety of colors.

According to one embodiment, the lip cosmetic composition according to one aspect of the present disclosure may not include a blue colorant.

According to one embodiment, the lip cosmetic composition may further include additional colorant different from the green colorant. The color of the additional colorant may be different from that of the green colorant.

According to one embodiment, it is sufficient if the additional colorant may be mixed with the green colorant to achieve a variety of colors, and the type is not limited.

According to one embodiment, the additional colorant may not be a blue colorant. According to one embodiment, the additional colorant may be a red colorant, for example, Red No. 227.

It is known that when a colorant is effectively dissolved and dispersed in the aqueous phase of an oil-in-water or water-dispersible composition, the color development and staining effect may be maximized. Meanwhile, titanium dioxide (TiO₂), which is primarily used to achieve a white color, tends to aggregate and/or precipitate in water, making it difficult to disperse. When titanium dioxide (TiO2) is used with other colorants, it does not fuse with the other colorants and becomes separated, leading to a messy tangled appearance when applied to the human body such as skin and lips. A cosmetic composition according to one aspect of the present disclosure, by including titanium dioxide coated with a hydrophilic compound, may exhibit excellent dispersibility and stability in water, preventing reaggregation and precipitation. It may show stability even in oil-in-water emulsion formulations, may be used in excess, and ultimately enables the development of various colors while enhancing the staining effect. In addition, a cosmetic composition according to one aspect of the present disclosure, by including titanium dioxide coated with a hydrophilic compound, may be mixed with the green colorant without separation, allowing for a clean makeup look when applied to the skin or lips.

According to one embodiment, the green colorant may be included in the cosmetic composition in an amount of 0.001 wt % to 5 wt % based on the total weight of the composition, but is not limited thereto. For example, the green colorant may be included in the cosmetic composition in an amount based on the total weight of the composition of 0.001 wt % or more, 0.01 wt % or more, 0.02 wt % or more, 0.03 wt % or more, 0.04 wt % or more, 0.05 wt % or more, 0.06 wt % or more, 0.07 wt % or more, 0.08 wt % or more, 0.09 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1 wt % or more, 2 wt % or more, 3 wt % or more, 4 wt % or more, 5 wt % or more, 0.001 wt % or less, 0.01 wt % or less, 0.02 wt % or less, 0.03 wt % or less, 0.04 wt % or less, 0.05 wt % or less, 0.06 wt % or less, 0.07 wt % or less, 0.08 wt % or less, 0.09 wt % or less, 0.1 wt % or less, 0.2 wt % or less, 0.3 wt % or less, 0.4 wt % or less, 0.5 wt % or less, 0.6 wt % or less, 0.7 wt % or less, 0.8 wt % or less, 0.9 wt % or less, 1 wt % or less, 2 wt % or less, 3 wt % or less, 4 wt % or less, 5 wt % or less, or any combination thereof.

According to one embodiment, the titanium dioxide coated with a hydrophilic compound may be titanium dioxide surface-treated with a hydrophilic compound. For example, the titanium dioxide coated with a hydrophilic compound may be in the form where the hydrophilic compound surrounds the surface of the titanium dioxide. According to one embodiment, the titanium dioxide coated with a hydrophilic compound may be obtained by spraying and mixing a hydrophilic compound (e.g., phytic acid) onto titanium dioxide, followed by heating. In this case, the mixing may involve stirring to combine the components. According to one embodiment, the titanium dioxide coated with a hydrophilic compound may be obtained through a reaction by vapor phase or a surface treatment method using a slurry.

“Hydrophilic compound” refers to a compound that has hydrophilic properties, and may be, for example, a hydrophilic organic compound or a hydrophilic inorganic compound. The hydrophilic compound may be a hydrophilic polymeric compound or a hydrophilic small molecule compound, but is not limited thereto.

According to one embodiment, the hydrophilic compound may be at least one selected from the group consisting of inositol, inositol salts, inositol phosphate esters, salts of inositol phosphate esters, alginic acid, and alginic acid salts.

According to one embodiment, the inositol phosphate ester may be at least one selected from the group consisting of inositol hexaphosphate, inositol pentaphosphate, inositol tetraphosphate, inositol triphosphate, inositol biphosphate, and inositol monophosphate, but is not limited thereto. Inositol hexaphosphate is also referred to as phytic acid.

According to one embodiment, the salt of the inositol phosphate ester may be at least one selected from the group consisting of salts of inositol hexaphosphate, salts of inositol pentaphosphate, salts of inositol tetraphosphate, salts of inositol triphosphate, salts of inositol biphosphate, and salts of inositol monophosphate, but is not limited thereto. The salt of inositol hexaphosphate may be phytate. Phytate may be in the form where phytic acid is bound to a metal salt (e.g., calcium, potassium, magnesium, etc.).

According to one embodiment, the hydrophilic compound may be at least one selected from the group consisting of phytic acid, phytic acid salts, alginic acid, and alginic acid salts. According to one embodiment, the hydrophilic compound may be at least one of phytic acid or sodium alginate.

According to one embodiment, the hydrophilic compound may have a hydrophile-lipophile balance (HLB) of 8 to 18, but is not limited thereto. For example, the hydrophilic compound may have an HLB of 8 or more, 9 or more, 10 or more, 11 or more, 12 or more, 13 or more, 14 or more, 15 or more, 16 or more, 17 or more, 18 or more, 8 or less, 9 or less, 10 or less, 11 or less, 12 or less, 13 or less, 14 or less, 15 or less, 16 or less, 17 or less, 18 or less, or any combination of these ranges.

In this specification, “HLB” refers to the hydrophile-lipophile balance, which is calculated according to Equation 1 below based on Griffin's method and is expressed as a value of 0 to 20. The larger the HLB value, the greater the hydrophilicity.

According to one embodiment, the titanium dioxide coated with a hydrophilic compound may be included in the cosmetic composition in an amount of 0.05 wt % to 10 wt % based on the total weight of the cosmetic composition, but is not limited thereto. For example, the titanium dioxide coated with a hydrophilic compound may be included in the cosmetic composition in an amount based on the total weight of the cosmetic composition of 0.05 wt % or more, 0.1 wt % or more, 0.2 wt % or more, 0.3 wt % or more, 0.4 wt % or more, 0.5 wt % or more, 0.6 wt % or more, 0.7 wt % or more, 0.8 wt % or more, 0.9 wt % or more, 1 wt % or more, 1.5 wt % or more, 2 wt % or more, 2.5 wt % or more, 3 wt % or more, 3.5 wt % or more, 4 wt % or more, 4.5 wt % or more, 5 wt % or more, 5.5 wt % or more, 6 wt % or more, 6.5 wt % or more, 7 wt % or more, 7.5 wt % or more, 8 wt % or more, 8.5 wt % or more, 9 wt % or more, 9.5 wt % or more, 10 wt % or more, 0.05 wt % or less, 0.1 wt % or less, 0.2 wt % or less, 0.3 wt % or less, 0.4 wt % or less, 0.5 wt % or less, 0.6 wt % or less, 0.7 wt % or less, 0.8 wt % or less, 0.9 wt % or less, 1 wt % or less, 1.5 wt % or less, 2 wt % or less, 2.5 wt % or less, 3 wt % or less, 3.5 wt % or less, 4 wt % or less, 4.5 wt % or less, 5 wt % or less, 5.5 wt % or less, 6 wt % or less, 6.5 wt % or less, 7 wt % or less, 7.5 wt % or less, 8 wt % or less, 8.5 wt % or less, 9 wt % or less, 9.5 wt % or less, 10 wt % or less, or any combination of these ranges.

When the content of titanium dioxide coated with a hydrophilic compound is less than 0.05 wt %, the white color is not effectively developed, making it difficult to achieve a neutral color tone. When it exceeds 10 wt %, there is a problem of titanium dioxide precipitating or caking phenomenon occurring within the composition.

The “caking phenomenon” may refer to the phenomenon of solidifying into a lump. When the caking phenomenon occurs in the composition, it may indicate poor dispersibility of the components included in the composition.

According to one embodiment, the lip cosmetic composition may be an oil-in-water (O/W) or water-dispersible formulation, but is not limited thereto.

A lip cosmetic composition according to one aspect of the present disclosure, when formulated in an oil-in-water or water-dispersible form, may allow the green colorant and/or titanium dioxide coated with a hydrophilic compound to be well dispersed in the water phase. When the green colorant and/or titanium dioxide coated with a hydrophilic compound are well dispersed in the water phase, a variety of colors, especially various staining colors, may be achieved, and the staining effect may be enhanced.

According to one embodiment, the lip cosmetic composition may be formulated into at least one product selected from the group consisting of lip tints, lipsticks, lip balms, lip creams, lip pencils, liquid rouges, and lip glosses.

One aspect of the present disclosure may provide a lip cosmetic including a green colorant and titanium dioxide (TiO₂) coated with a hydrophilic compound. As the green colorant and titanium dioxide coated with a hydrophilic compound have been described above, further detailed explanation will be omitted.

According to one embodiment, the lip cosmetic may be at least one selected from the group consisting of lip tints, lipsticks, lip balms, lip creams, lip pencils, liquid rouges, and lip glosses.

Using the lip cosmetic composition and/or method according to one aspect of the present disclosure, colors of various brightness and saturation may be achieved. The lip cosmetic composition according to one aspect of the present disclosure may achieve a variety of colors within a range that is difficult to implement with conventional blue colorants.

The lip cosmetic composition according to one aspect of the present disclosure may enable a clear color expression when applied to the lips, and darkening may not occur even after a predetermined period of time.

The lip cosmetic composition according to one aspect of the present disclosure may be applied to the lips without causing the yoplait phenomenon, providing a clean expression.

The lip cosmetic composition according to one aspect of the present disclosure may achieve colors of various brightness and saturation, provide a clear color expression when applied to the lips, prevent darkening, and simultaneously prevent the occurrence of the yoplait phenomenon (whitening phenomenon) on the lips.

The “yoplait phenomenon” may refer to the phenomenon where some components of the cosmetic composition, when applied to the lips, react with saliva or other substances, and separate on the lips, resulting in a whitish appearance. Yoplait phenomenon can also be referred to as phenomenon of “white ring”.

One aspect of the present disclosure provides a method for lip makeup including: applying the lip cosmetic composition described above.

One aspect of the present disclosure provides a method for lip coloring that includes: applying the lip cosmetic composition described above.

According to one embodiment, applying the lip cosmetic composition may comprise applying the lip cosmetic composition to the lips of a subject.

As the lip cosmetic composition has been described above, further detailed explanation will be omitted.

Using the method for lip makeup and/or method for lip coloring according to one aspect of the present disclosure, colors of various brightness and saturation may be achieved, a clear color expression may be provided on the lips, darkening may not occur, and simultaneously, the occurrence of the yoplait phenomenon (whitening phenomenon) on the lips may be prevented.

One aspect of the present disclosure provides a use of the lip cosmetic composition described above for preparing a cosmetic composition for lip makeup.

As the lip cosmetic composition has been described above, further detailed explanation will be omitted.

Hereinafter, the present invention will be described in detail with reference to Examples to specifically describe the present invention. The following examples are provided solely for illustrative purposes to aid in understanding the present invention, and are not intended to limit the scope and range of the present invention.

### 1. Lip Cosmetic Composition Preparation

Cosmetic compositions for Examples 1 to 2 and Comparative Examples 1 to 4 were prepared with the composition shown in Table 1 below (Units in Table 1: wt %). Specifically, the colorant components in Table 1 were dispersed and mixed in the aqueous components and then heated to 60° C. In this case, since the color strength of Green No. 201 is one-third of that of Blue No. 1, Green No. 5 was used at three times the content of Blue No. 1 to match the color tone similarly, and the composition was prepared. Then, the oil phase components were gradually added and mixed. The powder was mixed for 5 minutes at a speed of 1000 to 3000 rpm using a mixer after emulsification. Afterward, cooling was performed, and the composition was fully degassed to prepare an oil-in-water type makeup cosmetic composition.

TABLE 1

| | | | | | Comparative | Comparative | Comparative | Comparative |
|---|---|---|---|---|---|---|---|---|
| Division | Components | | Example 1 | Example 2 | Example 1 | Example 2 | Example 3 | Example 4 |
| Oil phase | Caprylic/Capric | | 20 | 20 | 20 | 20 | 20 | 20 |
| component | Triglyceride | | | | | | | |
| | Dicapryl carbonate | | 10 | 10 | 10 | 10 | 10 | 10 |
| | Polysorbate 60 | | 3 | 3 | 3 | 3 | 3 | 3 |
| Aqueous | Purified water | | Remaining | Remaining | Remaining | Remaining | Remaining | Remaining |
| component | | | amount | amount | amount | amount | amount | amount |
| | Glycerin | | 5 | 5 | 5 | 5 | 5 | 5 |
| | Disodium EDTA | | 0.05 | 0.05 | 0.05 | 0.05 | 0.05 | 0.05 |
| | Polyacrylate | | 0.5 | 0.5 | 0.5 | 0.5 | 0.5 | 0.5 |
| | crosspolymer-6 | | | | | | | |
| | Colorant | Red No. | 0.1 | 0.1 | 0.1 | 0.1 | 0.1 | 0.1 |
| | | 227 | | | | | | |
| | | Green No. | 0.03 | 0.03 | — | 0.03 | 0.03 | 0.03 |
| | | 201 | | | | | | |
| | | Blue No. 1 | — | — | 0.01 | — | — | — |

| | | | | | | | |
|---|---|---|---|---|---|---|---|
| Powder | Titanium dioxide*phytic | 1 | — | 1 | — | — | — |
| | acid | | | | | | |
| | Titanium | — | 1 | — | — | — | — |
| | dioxide*sodium alginate | | | | | | |
| | Titanium dioxide*silica | — | — | — | 1 | — | — |
| | Titanium | — | — | — | — | 1 | — |
| | dioxide*triethoxycaprylylsilane | | | | | | |
| | Titanium Dioxide | — | — | — | — | — | 1 |

In Table 1, titanium dioxide*phytic acid refers to titanium dioxide coated with phytic acid, titanium dioxide*sodium alginate refers to titanium dioxide coated with sodium alginate, and titanium dioxide*triethoxycaprylylsilane refers to titanium dioxide coated with an alkylsilane, specifically titanium dioxide coated with triethoxycaprylylsilane. Titanium dioxide*phytic acid was prepared by spraying the hydrophilic phytic acid onto titanium dioxide, then adding it to the mixer and stirring to ensure thorough mixing. Subsequently, through a heating process, the surface-treated powder coated with phytic acid (titanium dioxide*phytic acid) was obtained. Titanium dioxide*sodium alginate was prepared in the same manner as titanium dioxide*phytic acid, except that sodium alginate was used instead of phytic acid. Titanium dioxide*triethoxycaprylylsilane was prepared in the same manner as titanium dioxide*phytic acid, except that triethoxycaprylylsilane was used instead of phytic acid.

Additionally, in Table 1, titanium dioxide*silica refers to silica-complexed titanium dioxide, which is commonly and commercially used as water-dispersible titanium dioxide (Product Name: SIH-5 TiO₂ R250/Manufacturer: Daito Kasei).

### 2. Evaluation of Dispersibility, Formulation Stability, and Yoplait Phenomenon on the Lips Based on the Form of Titanium Dioxide

### (1) Dispersibility Evaluation

1 g of each cosmetic composition from Examples 1 to 2 and Comparative Examples 2 to 4 was mixed with 99 g of water, and dispersibility was immediately checked right after mixing. Additionally, the sedimentation rate and the occurrence of caking were evaluated after mixing.

TABLE 2

| Evaluation | | | Comparative | Comparative | Comparative |
|---|---|---|---|---|---|
| Standard | Example 1 | Example 2 | Example 2 | Example 3 | Example 4 |
| Immediate | Immediately | Immediately | Immediately | Aggregated | Immediately |
| dispersibility | dispersible | dispersible | dispersible | immediately | dispersible |
| | | | | after mixing, | |
| | | | | unable to | |
| | | | | disperse | |
| Sedimentation | Very slow | Medium | Slow | Not | Very fast (all |
| rate | (precipitation | (precipitation | (precipitation | measurable | precipitated |
| | occurs after | occurs after | occurs after | | within 10 |
| | 12+ hours) | 1 hour) | 6 hours) | | minutes) |
| Occurrence | X | X | Δ (Some | Not | ◯ |
| of caking | | | visible) | measurable | |

As shown in Table 2, Comparative Example 4, which includes non-coated titanium dioxide, was immediately dispersible, but it quickly precipitated, and caking phenomenon occurred. Comparative Example 3, which includes alkylsilane-coated titanium dioxide, aggregated immediately after mixing, and dispersion did not occur at all. Additionally, Comparative Example 2, which includes silica-complexed titanium dioxide, was immediately dispersible, and the sedimentation rate was slow. However, some caking phenomenon was observed.

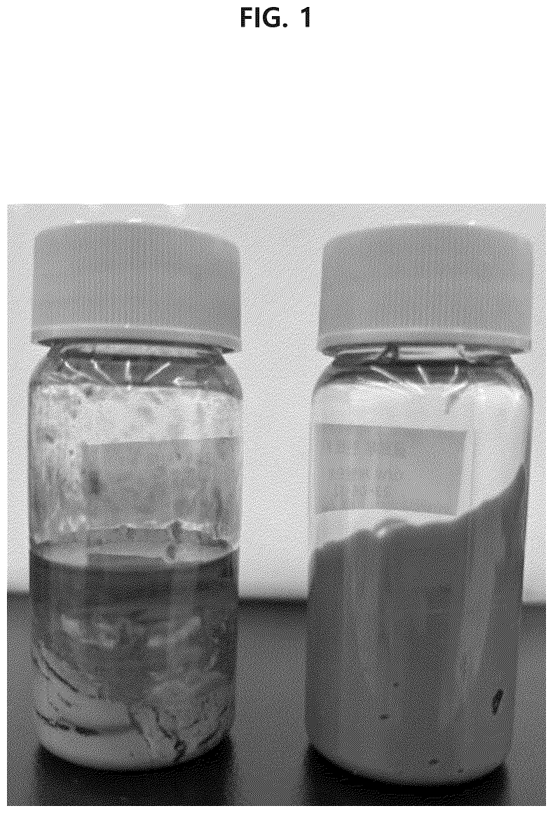

Additionally, a composition was further prepared in the same manner as Example 1, but with the content of titanium dioxide*phytic acid exceeding 10 wt %. The dispersibility of this composition was then evaluated. As a result, precipitation or caking easily occurred when more than 10% titanium dioxide*phytic acid was included in the composition (see the left container in FIG. 1).

### (2) Evaluation of Formulation Stability and Occurrence of Yoplait Phenomenon on the Lips

The cosmetic compositions of Example 1 and Comparative Example 2 were stored in a 55° C. constant temperature chamber, and the formulation stability was checked after 3 days. Additionally, the cosmetic compositions of Example 1 and Comparative Example 2 were applied to the lips, and the occurrence of the yoplait phenomenon was checked.

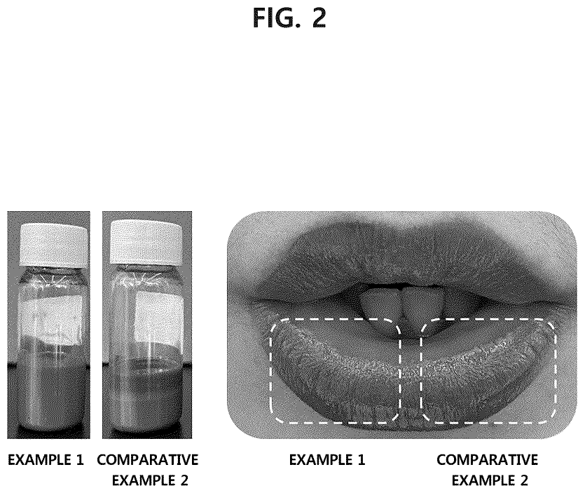

In Example 1, which used phytic acid-coated titanium dioxide, the dispersibility of the composition was maintained even after 3 days in the constant temperature chamber, and no formulation separation occurred. Meanwhile, Comparative Example 2, which used silica-complexed titanium dioxide, experienced formulation separation (see the left image in FIG. 2). Additionally, Example 1, which used phytic acid-coated titanium dioxide, applied cleanly to the lips. In contrast, Comparative Example 2, which used silica-complexed titanium dioxide, separated into a whitish appearance on the lips, resulting in an unclean color expression. This phenomenon appears to have occurred due to the easy clumping of titanium dioxide on the lips (see the right image in FIG. 2). The yoplait phenomenon on the lips appears to be related to the formulation stability.

### 3. Evaluation of Color of Cosmetic Composition Based on the Presence or Absence of Green Colorant

The color of the cosmetic compositions of Example 1 and Comparative Example 1, as well as the color after staining, were compared. As described in “1. Lip Cosmetic Composition Preparation” above, since the color strength of the green colorant is one-third of that of the blue colorant among the colorants, the content of Green No. 5 was used at three times the content of Blue No. 1 to match the color tone similarly, and the composition was prepared.

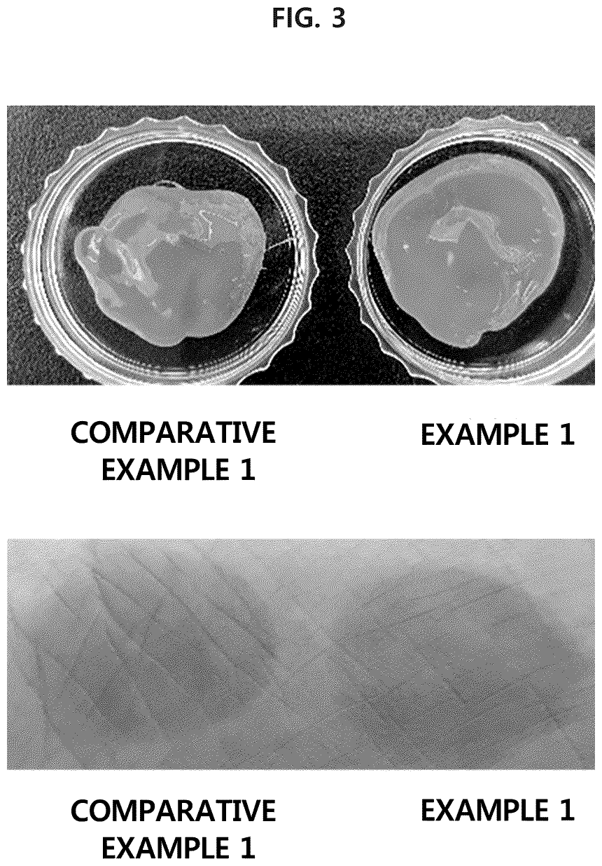

When visually observed, Example 1 and Comparative Example 1 showed similar color strengths. However, Example 1, which used the green colorant, was found to exhibit a relatively more yellowish, transparent color tone (see the upper image in FIG. 3). When comparing the colors after staining, Example 1, which used the green colorant, was able to achieve a non-dull coloring effect. Meanwhile, Comparative Example 1, which used the blue colorant, exhibited a relatively dull color (see the lower image in FIG. 3). This indicates that the green colorant has an excellent function in preventing color darkening.

### 4. Evaluation of the Ability to Implement Various Colors Based on the Presence or Absence of Green Colorant

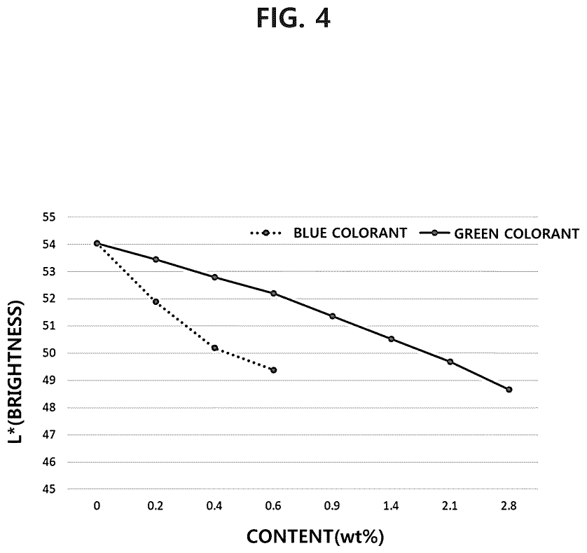

By adjusting the contents of the green colorant and blue colorant, it was confirmed that various colors can be achieved when using the green colorant. Specifically, the cosmetic composition was prepared by adjusting the content of Green No. 201 in the same manner as Example 1 and adjusting the content of Blue No. 1 in the same manner as Comparative Example 1. The degree of brightness reduction of the prepared cosmetic compositions was then measured using a colorimeter (SCI, Device Name: CM-5, Manufacturer: Konica Minolta). As a result, when using the blue colorant, even a small amount caused a significant decrease in brightness, making it difficult to achieve fine and precise color blending. Meanwhile, when using the green colorant, colors of various brightness levels could be achieved, compared to using the blue colorant. As shown in FIG. 4, the slope values of the brightness/content graph based on the colorimeter measurement results were approximately −7.7 when using the blue colorant, and approximately −3.1 when using the green colorant. This indicates that when using the green colorant, color blending can be done more than twice as precisely (see FIG. 4). Additionally, it can be observed that to match the brightness of the final product (e.g., cosmetic composition), the green colorant needs to be used at approximately 3 to 4 times the amount of the blue colorant.

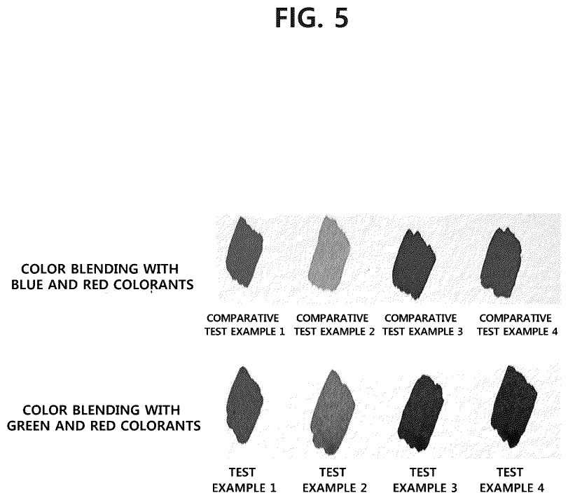

In addition, as a visible result, it was confirmed that when using the green colorant, a wider variety of toned-down, subdued colors can be achieved. Specifically, first, four color blending trials were conducted using the blue colorant and red colorant (Comparative Test Examples 1 to 4 in FIG. 5). The four color blending tests using the blue colorant were conducted under the same conditions, with only the content of the blue colorant being adjusted in small amounts. Additionally, four color blending trials were conducted using the green colorant and red colorant (Test Examples 1 to 4 in FIG. 5). The four color blending tests using the green colorant were conducted under the same conditions, with the content of the green colorant being adjusted in small amounts. Meanwhile, the four color blending tests using the green colorant were conducted under the same conditions as the four color blending tests using the blue colorant, but with the green colorant being used at three times the amount (weight) instead of the blue colorant. That is, in FIG. 5, Test Example 1 was color blended in the same manner as Comparative Test Example 1, but with the green colorant used at three times the content instead of the blue colorant. Additionally, Test Example 2 was color blended in the same manner as Comparative Test Example 2, but with the green colorant used at three times the content instead of the blue colorant. Test Example 3 was color blended in the same manner as Comparative Test Example 4, but with the green colorant used at three times the content instead of the blue colorant. Test Example 4 was color blended in the same manner as Comparative Test Example 4, but with the green colorant used at three times the content instead of the blue colorant. This was set based on the experimental results described above, which confirmed that to match the brightness of the final product, the green colorant needs to be used at approximately 3 to 4 times the amount of the blue colorant.

It was confirmed that when using the green colorant to tone down the color, a wider variety of colors can be achieved compared to using the blue colorant. When using the green colorant, it was possible to achieve toned-down, subdued colors (such as warm wine colors, subdued purple toned colors, etc.), whereas achieving subdued colors was not as easy when using the blue colorant (see FIG. 5). Green, being complementary to red, appears to enable the achievement of more subdued and warm colors.

### 5. Panel Evaluation

A panel consisting of 20 female subjects aged 25 to 39 was asked to evaluate the color expression and color longevity when the cosmetic compositions from the examples and comparative examples were applied to the lips. The evaluation was conducted on a 5-point scale, with higher scores given for better effectiveness in each item. The average scores of the 20 panelists for each item are shown.

As shown in Table 3, when the green colorant was used, the color was not only expressed clearly and attractively, but also no color darkening occurred. Meanwhile, when the blue colorant was used instead of the green colorant (Comparative Example 1), color darkening occurred after a predetermined period of time, resulting in dull lips. Additionally, when titanium dioxide coated with alkyl silane was used instead of titanium dioxide coated with a hydrophilic compound (Comparative Example 2), the color expression on the lips was not clean, resulting in lower scores for the item evaluating whether to be blended well with the lips.

TABLE 3

| Evaluation | | | Comparative | Comparative | Comparative | Comparative |
|---|---|---|---|---|---|---|
| Standard | Example 1 | Example 2 | Example 1 | Example 2 | Example 3 | Example 4 |
| Color is clear | 5 | 5 | 5 | 3 | 2 | 2 |
| and attractive | | | | | | |
| Lip expression | 5 | 4 | 4 | 2 | 1 | 2 |
| is clean | | | | | | |
| Blends well | 5 | 4 | 2 | 2 | 1 | 1 |
| with my lips | | | | | | |
| No color darkening | 5 | 4 | 2 | 4 | 1 | 1 |
| after 2 hours | | | | | | |

We claim:

1. A lip cosmetic composition, comprising:
an aryl-based green colorant; and
titanium dioxide (TiO₂) coated with a hydrophilic compound.

2. The lip cosmetic composition of claim 1, wherein the aryl-based green colorant is at least one selected from the group consisting of anthraquinone-based green pigments, triphenylmethane-based green pigments, and pyrene-based green pigments.

3. The lip cosmetic composition of claim 1, wherein the green colorant is at least one selected from the group consisting of Green No. 3 (CI number 42053), Green No. 201 (CI number 61570), Green No. 202 (CI number 61565), and Green No. 204 (CI number 59040).

4. The lip cosmetic composition of claim 1, wherein the hydrophilic compound is at least one selected from the group consisting of inositol, inositol salts, inositol phosphate esters, salts of inositol phosphate esters, alginic acid, and alginic acid salts.

5. The lip cosmetic composition of claim 1, wherein the hydrophilic compound is at least one of phytic acid or sodium alginate.

6. The lip cosmetic composition of claim 1, wherein the hydrophilic compound has an HLB of 8 to 18.

7. The lip cosmetic composition of claim 1, wherein the titanium dioxide coated with the hydrophilic compound is included in an amount of 0.05 to 10 wt %, based on a total weight of the composition.

8. The lip cosmetic composition of claim 1, wherein the lip cosmetic composition is an oil-in-water or water-dispersible formulation.

9. The lip cosmetic composition of claim 1, wherein the lip cosmetic composition is formulated into at least one product selected from the group consisting of lip tints, lipsticks, lip balms, lip creams, lip pencils, liquid rouges, and lip glosses.

10. The lip cosmetic composition of claim 1, further comprising:
additional colorant different from the green colorant, wherein the additional colorant is not a blue colorant.

11. A method for lip makeup, comprising:
applying the lip cosmetic composition according to claim 1.
